SEQUENCE LISTING (42 sequences)

SEQ ID NO: 1 - DNA, 894 bp, Dermatophagoides pteronyssinus
  CDS (1)..(894)
cgtccatcttccatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 2 - protein, 298 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 3 - DNA, 894 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccagcctccttgcatacgaatgggagtgacagttcgcatttgtcgcaaatcgatttcagctggagcatttccattgatactgcaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatggaagatggacg

SEQ ID NO: 4 - DNA, 654 bp, Dermatophagoides pteronyssinus
  CDS (1)..(654)
actaacgcctgcagtatcaatggaaatgctccagctgaaatcgatttgcgacaaatgcgaactgtcactcccattcgtatgcaaggaggctgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 5 - protein, 218 aa, Dermatophagoides pteronyssinus
TNACSINGNAPAEIDLRQMRTVTPIRMQGGWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 6 - DNA, 654 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccagcctccttgcatacgaatgggagtgacagttcgcatttgtcgcaaatcgatttcagctggagcatttccattgatactgcaggcgttagt

SEQ ID NO: 7 - DNA, 906 bp, Dermatophagoides pteronyssinus
  CDS (1)..(906)
cgtccatcttccatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 8 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 9 - DNA, 906 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatggaagatggacg

SEQ ID NO: 10 - DNA, 906 bp, Dermatophagoides pteronyssinus
  CDS (1)..(906)
cgtccatcttccatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctctggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 11 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGSGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 12 - DNA, 906 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccagagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatggaagatggacg

SEQ ID NO: 13 - DNA, 906 bp, Dermatophagoides pteronyssinus
  CDS (1)..(906)
cgtccatcttccatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctctggttcatgttgggctttctctggtgttgccgcaactgaatcagcttatttggcttaccgtaatcaatcattggatcttgctgaacaagaattagtcgattgtgcttcccaacacggttctcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 14 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGSGSCWAFSGVAATESAYLAYRNQSLDLAEQELVDCASQHGSHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 15 - DNA, 906 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgagaaccgtgttgggaagcacaatcgactaattcttgttcagcaagatccaatgattgattacggtaagccaaataagctgattcagttgcggcaacaccagagaaagcccaacatgaaccagagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatggaagatggacg

SEQ ID NO: 16 - DNA, 888 bp, Dermatophagoides pteronyssinus
  CDS (1)..(888)
cgtccatcttccatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgcagaagaccaaatgcacaaagattcggtatctcaaactattgccaaatttacccaccaaatgcgaacaaaattcgtgaagctttggctcaaacccacagcgctattgccgtcattattggcatcaaagatttagacgcattcacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttaatgatgattgaagaatatccatacgttgtcattttg

SEQ ID NO: 17 - protein, 296 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFTIIQRDNGYQPNYHAVNIVGYSNAQGVDYWIVRNSWDTNWGDNGYGYFAANIDLMMIEEYPYVVIL

SEQ ID NO: 18 - DNA, 888 bp, Dermatophagoides pteronyssinus
caaaatgacaacgtatggatattcttcaatcatcattaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgtgaatgcgtctaaatctttgatgccaataatgacggcaatagcgctgtgggtttgagccaaagcttcacgaattttgttcgcatttggtgggtaaatttggcaatagtttgagataccgaatctttgtgcatttggtcttctgcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactgcaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatggaagatggacg

SEQ ID NO: 19 - DNA, 909 bp, Dermatophagoides pteronyssinus
  CDS (1)..(909)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattcccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 20 - protein, 303 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYSQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 21 - DNA, 909 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttgggaatagtttgagataccgaaacgttgtgcatttggtcgtcggcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 22 - DNA, 897 bp, Dermatophagoides pteronyssinus
  CDS (1)..(897)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 23 - protein, 299 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 24 - DNA, 897 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccagcctccttgcatacgaatgggagtgacagttcgcatttgtcgcaaatcgatttcagctggagcatttccattgatactgcaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 25 - DNA, 897 bp, Dermatophagoides pteronyssinus
  CDS (1)..(897)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgggctttctctggtgttgccgcaactgaatcagcttatttggcttaccgtaatcaatcattggatcttgctgaacaagaattagtcgattgtgcttcccaacacggttctcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 26 - protein, 299 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGWAFSGVAATESAYLAYRNQSLDLAEQELVDCASQHGSHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 27 - DNA, 897 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgagaaccgtgttgggaagcacaatcgactaattcttgttcagcaagatccaatgattgattacggtaagccaaataagctgattcagttgcggcaacaccagagaaagcccagcctccttgcatacgaatgggagtgacagttcgcatttgtcgcaaatcgatttcagctggagcatttccattgatactgcaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 28 - DNA, 891 bp, Dermatophagoides pteronyssinus
  CDS (1)..(891)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattcacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 29 - protein, 297 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFTIIQRDNGYQPNYHAVNIVGYSNAQGVDYWIVRNSWDTNWGDNGYGYFAANIDLMMIEEYPYVVIL

SEQ ID NO: 30 - DNA, 891 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgtgaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactgcaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 31 - DNA, 909 bp, Dermatophagoides pteronyssinus
  CDS (1)..(909)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 32 - protein, 303 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 33 - DNA, 909 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 34 - DNA, 909 bp, Dermatophagoides pteronyssinus
  CDS (1)..(909)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctctggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 35 - protein, 303 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGSGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 36 - DNA, 909 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgacaaccgtgttgggaagcacaatcgactaattcttgttcagcaagatccaatgattgattacggtaagccaaataagctgattcagttgcggcaacaccagagaaagcccaacatgaaccagagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 37 - DNA, 909 bp, Dermatophagoides pteronyssinus
  CDS (1)..(909)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttctcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 38 - protein, 303 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGCGSCWAFSGVAATESAYLAYRNQSLDLAEQELVDCASQHGSHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 39 - DNA, 909 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgagaaccgtgttgggaagcacaatcgactaattcttgttcagcaagatccaatgattgattacggtaagccaaataagctgattcagttgcggcaacaccagagaaagcccaacatgaaccacagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

SEQ ID NO: 40 - DNA, 909 bp, Dermatophagoides pteronyssinus
  CDS (1)..(909)
atgcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctccagtatcaatggaaatgctccagctgaaatcgatttgcgacaaatgcgaactgtcactcccattcgtatgcaaggaggctctggttcatgttgggctttctctggtgttgccgcaactgaatcagcttatttggcttaccgtaatcaatcattggatcttgctgaacaagaattagtcgattgtgcttcccaacacggttctcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatacgttgtcatcctg

SEQ ID NO: 41 - protein, 303 aa, Dermatophagoides pteronyssinus
MRPSSIKTFEEYKKAFNKSYATFEDEEAARKNFLESVKYVQSNGGAINHLSDLSLDEFKNRFLMSAEAFEHLKTQFDLNAETNASSINGNAPAEIDLRQMRTVTPIRMQGGSGSCWAFSGVAATESAYLAYRNQSLDLAEQELVDCASQHGSHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 42 - DNA, 909 bp, Dermatophagoides pteronyssinus
caggatgacaacgtatggatattcttcaatcatcatcaaatcgatgttggcagcaaaataaccgtaaccattatcaccccaattggtatcccaactgtttcgtacgatccaataatcgacaccttgtgcgttactgtaaccaacaatgttgacagcgtgatagtttggttggtaaccattatcgcgttgaatgattgttcggccatcataatgacggaatgcgtctaaatctttgatgccaataatgacggcaatagcgctgtgggtttgagccaaagcttcacgaattttgtttacatttggtgggtaaatttggcaatagtttgagataccgaaacgttgtgcatttggtcgtcggcatgattgttctcgtgcaacgtatcgatagtagctttcttggacgacaccattatgttggatgtattcaataccacgtggaatggtatcaccatgagaaccgtgttgggaagcacaatcgactaattcttgttcagcaagatccaatgattgattacggtaagccaaataagctgattcagttgcggcaacaccagagaaagcccaacatgaaccagagcctccttgcatacgaatgggagtgacagttcgcatttgtcgcaaatcgatttcagctggagcatttccattgatactggaggcgttagtttcagcattcaaatcgaattgagttttgaggtgttcaaaagcttctgcactcatcaaaaatcggtttttgaattcatccaacgacaaatcggacaaatggttgatggcacctccatttgattgaacatattttactgattccaaaaagtttttacgggcagcttcttcatcttcgaaggtagcataacttttgttgaaggcttttttgtattcttcaaaagttttgatcgatgatggacgcat

(19) United States
(12) Patent Application Publication
(10) Pub. No.: US 2005/0053615 A1
(43) Pub. Date: Mar. 10, 2005

(54) Variants of mite group 1 allergens for the treatment of house dust mite allergy

(76) Inventors: Elaine A. Best, Ft. Collins, CO (US); Martin J. McDermott, Redwood City, CA (US)

Correspondence Address: HESKA CORPORATION;INTELLECTUAL PROPERTY DEPT., 1613 PROSPECT PARKWAY, FORT COLLINS, CO, 80525, US

(21) Appl. No.: 10/892,543

(22) Filed: Jul. 15, 2004

Related U.S. Application Data
(60) Provisional application No. 60/487,812, filed on Jul. 16, 2003.

(51) Int. Cl. A61K 39/00; C12N 5/06; C07K 14/435

(52) U.S. Cl. 424/185.1; 435/69.1; 435/348; 435/320.1; 530/350

(57) ABSTRACT

The present invention provides a method to produce variant recombinant mite Group 1 proteins with properties suitable for accelerated, specific immunotherapy. The variants of the present invention have greatly reduced IgE binding activity, but little or no loss in the ability to stimulate T cells in allergic individuals. The present invention also relates to a variant mite Group 1 protein obtained by such a method, and the use of such a variant mite Group 1 protein to reduce an allergic response to a mite Group 1 protein. The present invention also includes novel mite Group 1 nucleic acid molecules, proteins, recombinant molecules, and recombinant cells, as well as uses thereof.

### CROSS-REFERENCE TO RELATED APPLICATIONS

This application claims priority to U.S. Provisional Patent Application Ser. No. 60/487,812, filed Jul. 16, 2003, entitled “VARIANTS OF MITE GROUP 1 ALLERGENS FOR THE TREATMENT OF HOUSE DUST MITE ALLERGY,” which is incorporated herein by reference in its entirety.

### FIELD OF THE INVENTION

The present invention provides variant or mutant proteins of Group 1 house dust mite (HDM) allergens with properties suitable for accelerated, specific immunotherapy. The variants of the present invention have reduced IgE binding activity, but little or no loss in the ability to stimulate T cells in allergic individuals. The variant proteins are made by recombinant production in host cells transformed with mutant genes constructed by standard molecular biology techniques.

### BACKGROUND OF THE INVENTION

Type I allergic diseases, such as atopic dermatitis and atopic asthma, are induced by the cross-linking of mast cell-bound IgE to allergens. Diseases related to allergy and atopy affect a significant percentage of the population, including up to 20% of humans, and are increasing every year.

A significant proportion of type I allergic patients are mite allergic. For example, based on skin tests, at least 75% of the estimated 50 million asthmatics in the United States are mite allergic. The house dust mites Dermatophagoides farinae and Dermatophagoides pteronyssinus from Dermatophagoides sp. (designated herein as D. farinae and D. pteronyssinus) are the most common mites in the United States. These mites produce several classes, or groups, of allergens, one of which is known as Group 1 proteins, which are also found in other mite species. For example, considerable cross-reactivity has been found among Blomia tropicalis, D. farinae and Lepidoglyphus destructor allergens; see, for example, Colloff, 1992, Experimental and Applied Acarol. 16(1-2), 165-180; see also Arlian et al., 1993, J Allergy Clin Immunol 91, 1042-1050. Additionally, Group 1 proteins have been found in D. pteronyssinus, D. farinae, Euroglyphus maynei, and L. destructor; see, for example, Thomas et al., 1998, Allergy 53, 821-832. In human populations that are mite allergic, approximately 80% to 90% have IgE that is reactive to Group 1 proteins; see Thomas, 1996, Adv Exp Med Biol 409, 85-93. Thus, mite proteins, particularly Group I proteins, comprise an important allergen in type I allergic disease.

Mite Group 1 proteins share significant homology with a family of cysteine proteases including actinidin, papain, cathepsin H and cathepsin B. Group 1 proteins from different mites are highly homologous, approximately 25-kilodalton (kD) secretory glycoproteins, and are synthesized by the cell as a pre-pro-protein that is processed to a mature form. D. farinae, D. pteronyssinus, and E. maynei Group 1 proteins, for example, share about 80% identity. In particular, Group 1 proteins from D. farinae and D. pteronyssinus, also referred to as Der f 1 and Der p 1 proteins, respectively, show extensive cross-reactivity in binding IgE and IgG.

These Group 1 proteins are commonly found in the feces of mites and are thought to function as digestive enzymes in the mite intestine. Proteins found in high concentrations in the feces of house dust mites (HDM) (including Dernatophagoides spp., Euroglyphus maynei, Blomia tropicalis, and Lepidoglyphus destructor) are a contributing factor in IgE meditated type I allergic disease (perennial rhinitis, asthma, and atopic dermatitis) worldwide.

Since the early 1900's, HDM allergy has been managed by specific immunotherapy involving the systemic delivery of increasing doses of HDM extracts over an extended period of time. Although this treatment is effective in many patients, it has some disadvantages. 1) The allergen contents of various extracts may differ by as much as six-fold. Accordingly, some patients may respond poorly to specific immunotherapy because they are treated with allergen extracts that contain an inappropriate level (either sub- or super-optimal amounts) of the relevant allergen. 2) The risk of IgE-mediated anaphylaxis increases with the amount of antigen injected, and injections must be titrated over a long period of time until a “maximum tolerated dose” is established.

One strategy that has been used to alleviate some of the problems of earlier methods of immunotherapy has been the identification of T cell reactive peptides on the allergens and the use of such peptides in immunotherapy. This approach has the advantage that the peptides usually have little or no IgE binding epitopes, thus they are not able to induce the negative IgE-mediated side effects such as histamine release. The strategy is limited however, by the fact that different individuals in a population recognize different T cell epitopes on the allergen. Therefore, to produce a T cell peptide that is useful for immunotherapy for many individuals, it is necessary to produce a mixture of peptides, or a recombinant protein that links multiple peptides, while still maintaining the reduced IgE reactivity.

As an alternative approach, natural isoforms have been found that retained T cell reactivity, but did not retain IgE binding reactivity. Such isoforms have not been found for many allergens, however. When isoforms with the desired characteristics do not occur naturally, it may be possible to create such isoforms by genetically engineered variants.

A more rational type of specific immunotherapy would be the administration of hypoallergens, which are protein allergens designed to have low or no IgE-binding affinity, and normal or near normal T cell antigenicity. Site-directed mutagenesis of cysteine residues was used to construct mutants of house dust mite allergens Der p2 and Der f2. These mutations destroyed conformational IgE-binding epitopes (Smith et al., 1996, Mol. Immunol, 33:399-405; Takai et al., 1997, Nat. Biotech. 15:754-758). Genetically engineered variants of a timothy grass pollen allergen have also been constructed; these variants showed significantly reduced IgE reactivity but showed comparable proliferation stimulation of T cell clones and T cell lines (Schramm et al., 1999, Journal of Immunol. 162(4):2406-14). Prior to the present invention, the rational design of recombinant hypoallergens of the group I HDM allergens has been hampered because the literature concerning the immunologically relevant sequences, structures, or functions of the group 1 HDM allergens is not in complete agreement.

Because it has been difficult to isolate large quantities of pure, active Der p 1 or Der f 1, the crystal structures of the group 1 HDM allergens have not been solved; this information would facilitate the rational design of hypoallergenic variants. cDNA clones of these allergens have provided sequence information for modeling studies. The group 1 mite allergens from Dermatophagoides sp. have a 19 residue signal peptide, an 80 residue proenzyme sequence, and a 222 (Der p 1) or 223 (Der f 1) residue mature protein, a single N-linked glycosylation site (residues 52-54 of mature Der p 1 and residues 53-55 of mature Der f 1). Using the amino acid sequence of Der p 1 deduced from cDNA clones described by Chua et al., 1993, Int. Arch. Allergy Immunol. 101(4): 364-368 (also Thomas et al., 1988, Int. Arch. Allergy Immunol. 85(1):127-129) and known protein homologs (papain, actinidin, and papaya proteinase omega), Topham constructed a 3-dimensional model of Der p 1 (Topham et al., 1994, Protein Eng. 7(7):869-894). Based on its sequence similarity, other group I allergens, particularly Der f 1, are expected to have the same conformational structure as Der p 1.

The roles of various sequences, structures, or functions of the group 1 HDM allergens in type I allergic disease are poorly understood. The group 1 allergens are presumed to have structural and mechanistic features in common with other cysteine protease homologs. In particular, six cysteine residues are assumed to be involved in disulfide bridges (C4-C117, C31-71, and C65-C103 of mature Der p 1 and C4-C118, C32-C72, and C66-C104 of mature Der f 1). Hewitt et al., 2000, Clin. Exp. Allergy 30(6);784-793 demonstrated that Der p 1 is inhibited by serine as well as cysteine protease inhibitors. In addition, those investigators demonstrated that Der p 1 selectively cleaves the low-affinity receptor for human IgE (CD23) from IgE-secreting B cells. Based on these data, these investigators proposed that the proteolytic activity of the group 1 mite allergens may contribute to their allergenicity. The active site residues of Der p 1 are predicted to include Q28, G32, C34, H170, G170, and the sequence NSW at residues 190 to 192. The residues that form the active site and the disulfide bonds are highly conserved between Der p 1 and Der f 1.

The major IgE epitopes of the group 1 HDM allergens are expected to be solvent accessible, hydrophilic regions. Using a variety of methods (recombinant and synthetic peptides, phage display), several investigators have identified IgE epitopes on Der f 1 and Der p 1. Using linear, overlapping peptides, Jeannin et al., 1993, Mol. Immunol. 30(16): 1511-1518, identified residues 52-71, 117-133, 176-187, and 189-199 as major IgE epitopes on Der f 1. Using cyclic peptides displayed on the surface of phages, Furmonaviciene et al., 1999, Clin. Exp. Allergy 29(11):1563-1571, identified residues 147-160 as a “potential” IgE epitope of Der p 1. Residues 1-33, 60-94, 101-111, 155-187, and 209-222 have also been implicated in IgE binding (see references cited in Topham et al., 1994, Protein Eng. 7(7):869-894). Thus, there is no agreement in the literature on which residues constitute the major IgE epitopes. A general conclusion from these results is that the major IgE epitopes on the group 1 allergens are discontinuous and conformational.

The major T cell epitopes of the group 1 allergens are expected to be linear. Using overlapping peptides, several regions have been identified as important. These include residues 45-67, 94-104, 117-143, 101-143, 107-119, 110-119, and 110-131 (reviewed in O'Hehir et al., 1993, Eur. J. Clin. Invest. 23(12):763-772).

### SUMMARY OF THE INVENTION

The present invention describes methods for altering the conformation or sequence of the group 1 mite allergens so that IgE binding activity is reduced compared to the wild type allergen. These altered group 1 mite proteins have little or no alteration in their capacity to stimulate T cells.

The present invention provides a method to produce a recombinant mite Group 1 protein, wherein the protein has altered biological activity compared to the wild type protein. The proteins of the present invention are hypoallergens. The proteins of the present invention have low binding to IgE, preferably equal to or less than 100 fold the IgE binding activity of wild type protein. Thus, the mutant proteins have lost some B-cell epitopes. The preferred mutant proteins maintain the T-cell epitopes, that is, the mutant proteins retain the activity of causing proliferation of a T cell that proliferates in response to a native mite Group 1 protein.

The proteins of the present invention have particular use as therapeutics for treatment of allergy. The proteins of the present invention bind less IgE and the inflammatory response is lower than is the case with native or recombinant wild type allergens. However, since T cell proliferation is stimulated at the same rate as is the case with the wild type or native proteins, the therapeutic benefit is obtained.

In one embodiment of the invention, some or all of the cysteine residues involved in disulfide bonding are substituted with conservative residue substitutions (especially serine). Genetically modified group 1 mite allergens with changes that affect surface exposed residues are another embodiment of the invention.

The present invention also includes isolated novel nucleic acid molecules, recombinant molecules, and recombinant microorganisms that encode the proteins of the present invention:

The present invention builds on knowledge and findings described in related PCT publication WO 01/29078 A2, published Apr. 26, 2001, entitled “METHOD FOR THE PRODUCTION AND USE OF MITE GROUP 1 PROTEINS,” which is hereby incorporated by reference in its entirety.

Because individuals in a population may respond to different T cell epitopes on an antigen, the strategies outlined below preferably, but not necessarily, include the entire sequence of the antigen, and the full repertoire of T cell epitopes, rather than a peptide fragment. Nucleotide sequences of genes encoding group 1 mite proteins with changes that are predicted to reduce IgE reactivity are provided.

### DETAILED DESCRIPTION OF THE INVENTION

The present invention provides a method to produce a recombinant mite Group 1 protein that has the following functions (i.e., activities, properties): (a) decreased binding to IgE compared to a native mite Group 1 protein; and (b) causing proliferation of a T cell that proliferates in response to a native mite Group 1 protein.

Substitutions and deletions in the nucleic acid sequence are created by PCR or similar methods to systematically evaluate the contributions of the disulfide bonds to biological activity of the encoded variant. In particular, serine residues are substituted at the six cysteines residues believed to be involved in disulfide bonds, both singly and in pairs. (Other preferred amino acid exchanges include ones that have similar charge and size such that they do not disturb the protein structure, e.g., threonine, alanine, or valine.) These substitutions are made to provide mutant proteins that are altered in the desired manner. Removal of a single cysteine is expected to allow disulfide bond removal and possibly reshuffling. DNA sequences of the following examples are provided.

Mutations that reduce or eliminate cysteine protease activity are contemplated. For example, mutations (residue substitutions and deletions) that change C34 (mature Der p 1) and C35 (mature Der f 1), putative active site residues, are described in related PCT publication WO 01/29078A2, ibid. Mutations that alter the conformation of the protein are also postulated to affect cysteine protease function. Mutants with altered disulfide bonding may be affected in their ability to self-process. Mutations that affect the cysteine protease activity may negatively affect the self-processing ability of pro-Der p 1 or pro Der f 1. If necessary, following refolding, the pro-peptide may be enzymatically removed.

One embodiment of the present invention is a protein in which mutations have been introduced into the sequence in order to affect intra molecular disulfide bonding. Such mutations can be deletions or substitutions, with one or more amino acid residues being mutated or deleted. Preferred sites within the protein at which to create mutations are cysteine residues. Mutations in which one or more cysteine is substituted by a different amino acid or in which one or more cysteines are deleted are contemplated. Also contemplated are protein mutants in which one or more amino acid residue bordering the cysteine residues are deleted along with the cysteine residue. A useful cysteine to mutate is any cysteine involved in disulfide bonding. Particularly useful cysteine sites to mutate include C4 (the number refers to the position of the amino acid in the protein sequence) of mature Der p1, C31 of mature Der p1, C65 of mature Der p1, C71 of mature Der p 1, C65 of mature Der p 1 and C117 of mature Der p 1. Also useful are C4 of mature Der f1, C32 of mature Der f1, C66 of mature Der f1, C72 of mature Der f1, C104 of mature Der f1 and C118 of mature Der f1.

In addition, deletions or substitutions that remove or alter the sequence R151 to R156 of mature Der p 1 (R152 to R157 of mature Der f 1) are herein described. In particular, R151N or G, H152S, D154S, and R156N or G are substituted in mature Der p 1. (Corresponding substitutions are also made in mature Der f 1.)

One embodiment of the instant invention is a protein encoded by a nucleic acid molecule comprising a nucleic acid sequence selected from the group consisting of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37 and SEQ ID NO:40. One embodiment of the instant invention is a protein comprising an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38 and SEQ ID NO:41. Due to natural variability found in nature, it is appreciated that allelic variants of genes and their encoded proteins may exist. Such allelic variants have sequence changes in regions of the protein (other than the cysteine residues and the specific regions at which to engineer mutations described above) that do not substantially affect activity of the protein. A mutation that substantially affects a proteins activity causes an enhancement or a decrease of greater than about 10% compared to a protein in which such a sequence change is not made. Allelic variants typically have only a small percentage of their amino acids which differ from other Der p1 proteins. For example, an allelic variant of a Der p1 protein may differ in sequence from another Der p1 protein by only 1 amino acid, 2 amino acids, 3 amino acids, 4 amino acids, 5 amino acids, 6 amino acids, 7 amino acids, 8 amino acids, 9 amino acids or 10 amino acids. As noted above, these differences occur at sites different from (but possibly in addition to) the mutations engineered at the cysteine and arginine regions described above. The inventors also contemplate proteins comprising an amino acid sequence at least about 90%, at least about 95% or at least about 98% identical to an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38 and SEQ ID NO:41.

Recombinant proteins produced in E. coli accumulate in the cytoplasm in an insoluble or partially soluble form. Insoluble proteins are recovered from inclusion bodies and refolded into a monomeric form. Such proteins may include the pro-form or the mature form of the mite allergen. The preferred expression system for E. coli is an autonomously replicating plasmid that includes a selectable marker (e.g., an auxotrophic requirement or an antibiotic resistance phenotype) and an inducible promoter, (e.g., the rightward promoter of bacteriophage lambda (P_{R}) controlled by a thermosensitive repressor protein, cI857).

Proteins made using methylotrophic yeast hosts may include the pro-form or the mature form of the group 1 mite allergen, and be secreted into the culture medium, the cytoplasm, or a specific organelle (e.g., peroxisome). The preferred expression system for the methylotrophic yeast P. pastoris is an integrated DNA sequence(s) that contains either an inducible promoter (e.g., P. pastoris AOX1) or a constitutive promoter (P. pastoris GAP), a selectable marker (e.g., an auxotrophy or a drug resistance phenotype). The signal peptide from the native allergen or from another source (e.g., Saccharomyces cerevisiae α-mating pre-pro sequence, or P. pastoris PHO 1) may be fused to the recombinant allergen to direct the expressed protein into the culture medium. Similarly, a heterologous signal may be fused to the recombinant allergen to target it to the peroxisome.

One embodiment of the present invention is a composition that includes a mite Group 1 protein of the present invention and an excipient. Also included is a method to use such a composition to reduce an allergic response to a mite Group 1 protein in a mite-allergic animal. Such a method includes the step of administering such a composition to such an animal.

It is to be noted that the term “a” or “an” entity refers to one or more of that entity; for example, a protein refers to one or more proteins or at least one protein; as another example, a nucleic acid molecule refers to one or more nucleic acid molecules or at least one nucleic acid molecule. As such, the terms “a” or “an”, “one or more”, and “at least one” can be used interchangeably herein. It is also to be noted that the terms “comprising”, “including”, and “having” can be used interchangeably herein. According to the present invention, an isolated, or biologically pure, protein or nucleic acid molecule is a protein or nucleic acid molecule, respectively, that has been removed from its natural milieu. As such, “isolated” and/or “biologically pure” do not necessarily reflect the extent to which the protein or nucleic acid molecule has been purified. An isolated protein or nucleic acid molecule of the present invention can be obtained from its natural source, can be produced using recombinant nucleic acid technology, or can be produced by chemical synthesis.

A mite Group 1 protein refers to a Group 1 protein from (including derived from) a species of mite. A recombinant mite Group 1 protein of the present invention refers to a mite Group 1 protein produced using the techniques of recombinant nucleic acid technology. A suitable recombinant mite Group 1 protein of the present invention is a Group 1 protein from any species of mite that is produced using recombinant nucleic acid techniques. Such species include, but are not limited to, Acarus siro, Aleuroglyphus ovatus, Blomia kulagini, Blomia tropicalis, Chortoglyphus arcuatus, Dermatophagoides farinae, Dermatophagoides microceras, Dermatophagoides pteronyssinus, Euroglyphus maynei, Glycyphagus domesticus, Gohieria fusca, Lepidoglyphus destructor, Psoroptes ovis, Pterolichus obtusus, Sarcoptes scaiei, Tyrophagus longior, and Tyrophagus putrescentiae. Preferred mite Group 1 proteins of the present invention include those of the genera Blomia, Dermatophagoides, Euroglyphus, Lepidoglyphus, and Tyrophagus, with those of the species B. kulagini, B. tropicalis, D. farinae, D. microceras, D. pteronyssinus, E. maynei, L. destructor, and T. longior being more preferred. Particularly preferred mite Group 1 proteins are Dermatophagoides and Euroglyphus maynei Group 1 proteins, with D. farinae, D. pteronyssinus, and E. maynei Group 1 proteins being even more preferred.

A native mite Group 1 protein refers to a Group 1 protein recovered directly from a species of mite. In one embodiment, a native mite Group 1 protein is purified from a mite extract under conditions that retain the mite Group 1 protein's inherent IgE reactivity. As used herein, a protein's IgE reactivity refers to the ability of that protein to selectively or specifically bind IgE that is reactive with a mite Group 1 protein. As used herein, the terms selectively (or specifically) binds IgE and selectively (or specifically) binds to (or with) IgE refer to the ability of a mite Group 1 protein of the present invention to preferentially bind to IgE specific for Group 1 allergens, without being able to substantially bind to IgE specific for other allergens. Methods of measuring preferential binding are known to those skilled in the art. One example of a measure of preferential binding is avidity. Preferential binding is defined as a binding activity for one class of molecule at least about 2 times (2×), at least about 3×, at least about 4×, at least about 5×, at least about 10×, at least about 15× at least about 20×, at least about 50× or at least about 100× greater than the binding activity for a second class of molecule. IgE that is reactive with a mite Group 1 protein is an IgE antibody that reacts with a mite Group 1 protein in a manner equivalent to an IgE raised in response to a mite Group 1 protein. Methods to purify native mite Group 1 proteins such that they retain their inherent (i.e., natural) IgE reactivity are known to those skilled in the art, and are disclosed in PCT publication WO 01/29078 A2. Such a native mite Group 1 protein can be used as a “standard” by which to compare a function, or activity, of a mite Group 1 protein obtained by other means, such as by expression of a recombinant form of a mite Group 1 protein of the same species as that from which the native protein is purified.

The ability of a recombinant mite Group 1 protein to selectively bind to IgE can be assayed by methods known in the art, such as, but not limited to, those disclosed herein. Methods to compare IgE binding activity of the variant or mutant proteins of the present invention with the IgE binding activity of a native mite Group 1 protein are also known in the art and include, but are not limited to, those methods disclosed herein.

In one embodiment, recombinant variant and native forms of a mite Group 1 protein are contacted (i.e., reacted) with serum samples from animals that are allergic to mites using, for example, an ELISA format, and a determination is made of what percentage of serum samples that are reactive with the native protein are also reactive with the variant recombinant protein. Preferably the testing is conducted using assay conditions in which essentially all of the mite-allergic serum samples give a positive result with the native mite Group 1 protein. An example of how the percentage is determined is as follows: if a recombinant variant mite Group 1 protein is tested against 10 mite-allergic serum samples, wherein all 10 samples are reactive to a native mite Group 1 protein, and only 7 samples are reactive to the variant recombinant protein, the reactivity is expressed as 7/10, or 70%. That is, the variant recombinant mite Group 1 protein selectively binds to IgE of 70% of serum samples comprising IgE that selectively bind to a native mite Group 1 protein.

In another embodiment, the abilities of recombinant variant and native forms of a mite Group 1 protein to selectively bind to a monoclonal antibody raised against a native mite Group 1 native protein (i.e., an anti-native mite Group 1 monoclonal antibody) or to a panel of such monoclonal antibodies are compared. A recombinant Group 1 protein that has comparable, or substantially equivalent, activity to a native Group 1 protein is a recombinant mite Group 1 protein that reacts with essentially all of the monoclonal antibodies that react with the native mite Group 1 protein. In the addition, the binding affinities of the monoclonal antibodies for the recombinant Group 1 protein should be very similar to the respective binding affinities of the monoclonal antibodies for the native Group 1 protein. The binding affinity can be determined with a simple dose-response curve.

A preferred method to determine the IgE reactivity of a variant recombinant mite Group 1 protein is to compare the reactivities of variant proteins and the native forms of a mite Group 1 protein to IgE in serum samples that selectively bind to native mite Group 1 proteins. The phrase, an IgE activity substantially equivalent to that of a native mite Group 1 protein refers to an IgE reactivity that is very comparable, or similar to, the activity of a native mite Group 1 protein. By comparable, or similar, is meant an IgE activity with a variance of no more than about 10% compared to the activity of a native mite Group 1 protein. Preferred mite Group 1 variant proteins of the present invention exhibit IgE reactivities that are at most about 90%, preferably at most about 85%, preferably at most about 80%, preferably at most about 75%, preferably at most about 70%, preferably at most about 65%, preferably at most about 60%, preferably at most about 55%, preferably at most about 50%, preferably at most about 45%, preferably at most about 40%, preferably at most about 35%, preferably at most about 30%, preferably at most about 25%, preferably at most about 20%, preferably at most about 15%, preferably at most about 10%, preferably at most about 5%, preferably at most about 1% equivalent to a native mite Group 1 protein. A particularly preferred variant mite Group 1 protein of the present invention selectively binds to IgE of at most about 1% of serum samples comprising IgE that selectively bind to a native mite Group 1 protein.

The ability of a variant mite Group 1 protein of the present invention to cause proliferation of a T cell that proliferates in response to a native mite Group 1 protein, also referred to herein as T cell reactivity, can be assayed by methods known in the art; see, for example, Janeway, et al., 1996, Immunobiology, Second Edition, Garland Publishing Inc., New York, N.Y.; Janeway et al., ibid., which are hereby incorporated by reference in their entirety. In one embodiment, a variant mite Group 1 protein of the present invention preferably contains most or all of the relevant dominant T cell epitopes to stimulate T cell proliferation. In order to determine whether a variant mite Group 1 protein contains relevant dominant T cell epitopes, T cell proliferation assays can be performed, by methods known to those skilled in the art, and the ability of that variant mite Group 1 protein to stimulate T cell proliferation can be compared to the ability of the corresponding native mite Group 1 protein to stimulate T cell proliferation. A preferred recombinant mite Group 1 protein of the present invention stimulates T cell proliferation as well as, or in a manner comparable to, a native mite Group 1 protein. Other preferred recombinant mite Group 1 proteins of the present invention can have an altered ability to stimulate T-cell proliferation, for example, about 25%, preferably about 20%, preferably about 15%, preferably about 10%, preferably about 5% enhanced or reduced stimulating activity compared with a native mite Group I protein depending on the intended use.

A mite Group 1 nucleic acid molecule of the present invention encodes a variant protein of the present invention, and is produced using, for example, recombinant nucleic acid technology (e.g., polymerase chain reaction (PCR) amplification or cloning) or chemical synthesis. A nucleic acid molecule of the present invention can be DNA, RNA, or a derivative of DNA or RNA. Mite Group 1 nucleic acid molecules of the invention include natural forms including allelic variants that do not affect the IgE and T cell epitopes, nucleic acids optimized for expression in a particular host, complementary DNAs (cDNAs) or RNAs derived from genomic sequences (including those incorporating natural variations), and nucleic acid molecules modified by nucleotide insertions, deletions, substitutions, and/or inversions to produce the nucleic acids encoding the variant proteins of the present invention and can be produced using a number of methods known to those skilled in the art, see, for example, Sambrook et al., 1989, Molecular Cloning: A Laboratory Manual, Cold Spring Harbor Labs Press; Sambrook et al., ibid. is incorporated by reference herein in its entirety. For example, nucleic acid molecules can be modified using a variety of techniques such as site-directed mutagenesis, chemical treatment, restriction enzyme cleavage, ligation of nucleic acid fragments, PCR amplification, synthesis of oligonucleotide mixtures and ligation of mixture groups to “build” a mixture of nucleic acid molecules, and combinations thereof. In one embodiment, modifications are made to nucleic acid molecules encoding Group I proteins so the expressed protein contains conservative substitutions within its sequence. As used herein, the term “conservative substitutions” and the like, refer to the substitution of one amino acid within an amino acid sequence, by an amino acid having similar properties such as charge, size, hydrophobicity or cyclic structure of the side chain. For example, the substitution of an alanine, which has a small side chain, with a glycine, which also has a small side chain, represents a conservative substitution. Methods of grouping amino acids are known to those skilled in the art; see, for example, Darnell, et al., 1990, Molecular Cell Biology, Second Edition, Scientific American Books, which is incorporated by reference herein.

One embodiment of the present invention is a nucleic acid molecule comprising a nucleic acid sequence selected from the group consisting of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37 and SEQ ID NO:40. One embodiment of the instant invention is a nucleic acid molecule comprising a nucleic acid sequence encoding a protein comprising an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38 and SEQ ID NO:41.

A full-length mite Group 1 protein, i.e., the initial translation product, is a pre-pro-form of the protein containing a pre-segment and a pro-segment as well as the mature protein. The pre-segment, or pre-sequence, also known as the leader sequence or the signal sequence, apparently directs the mite Group 1 protein to be secreted from the cell and is proteolytically cleaved to yield a pro-form of the protein. The pro-segment, or pro-sequence, is then proteolytically cleaved to yield the mature Group 1 protein. A preferred protein to express is the pro-form (which includes the mature sequence as well as the pro sequence, but does not have a signal sequence attached) or the mature form. An example of a mature form of one Group I protein is SEQ ID NO:5 which begins at T81 of the pro-form (SEQ ID NO:2). Mature forms or Group I proteins, each starting at T81 and going to the carboxy terminal amino acid, are contemplated for all of the mutant sequences disclosed.

Nucleic acid molecules and proteins of the present invention that are of certain species and lengths are denoted as follows: a Der f 1 nucleic acid molecule protein of a certain length is denoted as nDerf1_{#}, for example, nDerf1₉₆₃, wherein “#” refers to the number of nucleotides in that molecule; in a similar fashion, a Der p 1 nucleic acid molecule of a certain length is denoted as nDerp1_{#}, a E. maynei Group 1 nucleic acid molecule of a certain length is denoted as nEurm1_{#}, and so on. Similarly, a Der f 1 protein of the present invention of known length is denoted PDerf1_{#}, a Der p 1 protein of the present invention of known length is denoted PDerp1 #, a E. maynei Group 1 protein of a certain length is denoted as PEurm1_{#}, and so on. The variant forms of the proteins are denoted as follows: ProDerp1_{ΔC31-34} is the propeptide and has a deletion of amino acids 31 through 34 (of the mature protein) and pro Der p1_{C4S} is the propeptide with a change from cysteine to serine at amino acid 4 of the mature protein.

One embodiment of a method to produce a variant mite Group 1 protein of the present invention includes the steps of (a) culturing a methyltrophic yeast microorganism transformed with a nucleic acid molecule encoding the variant mite Group 1 protein, and (b) recovering the variant mite Group 1 protein from the methyltrophic yeast microorganism. A methyltrophic yeast microorganism is a yeast strain capable of using methanol as its sole carbon source. Although any methyltrophic yeast can be used in the methods of the present invention, preferred methyltrophic yeast microorganisms to transform and culture include those of the genera Pichia, Hansenula, Torulopsis, and Candida, with the genus Pichia being particularly preferred. Preferred methyltrophic yeast species include Pichia pastoris, Pichia acaciae, Pichia anomala, Pichia augusta, Pichia capsulata, Pichia fabianii, Pichia farinosa, Pichia guilliermondii, Pichia methanolica, Pichia norvegensis, Pichia pinus, Pichia stipitis, Hansenula polymorpha, and Candida boidinii. A preferred Pichia microorganism is Pichia pastoris.

Another embodiment of a method to produce a variant mite Group 1 protein of the present invention includes the steps of (a) culturing an E. coli microorganism transformed with a nucleic acid molecule encoding the variant mite Group 1 protein under conditions in which the protein forms an inclusion body in the E. coli microorganism, (b) isolating the inclusion body from the E. coli microorganism, and (c) recovering the variant mite Group 1 protein from the inclusion body.

Transformation of a nucleic acid molecule of the present invention into a microorganism can be accomplished by any method by which a nucleic acid molecule can be inserted into the cell. Transformation techniques include, but are not limited to, transfection, electroporation, microinjection, lipofection, adsorption, and protoplast fusion. Transformed nucleic acid molecules of the present invention can remain extrachromosomal or can integrate into one or more sites within a chromosome of the transformed (i.e., recombinant) microorganism in such a manner that their ability to be expressed is retained. A transformed microorganism is also referred to herein as a transformed cell, a recombinant microorganism or a recombinant cell.

A microorganism to be transformed can be either an untransformed cell or a cell that is already transformed with at least one nucleic acid molecule (e.g., one or more nucleic acid molecules encoding one or more proteins of the present invention and/or other proteins). A recombinant microorganism of the present invention is preferably produced by transforming a host cell with one or more recombinant molecules comprising one or more nucleic acid molecules of the present invention.

As used herein, a recombinant molecule comprises a nucleic acid molecule of the present invention operatively linked to a transcription control sequence, preferably contained within an expression vector. The phrase operatively linked refers to joining of a nucleic acid molecule to a transcription control sequence in a manner such that the molecule is able to be expressed when transformed into a yeast or E. coli microorganism. As used herein, an expression vector is a DNA or RNA vector, typically either a plasmid or viral genome, that is capable of transforming a cell and of effecting expression of a specified nucleic acid molecule. A preferred recombinant molecule of the present invention contains regulatory sequences such as transcription control sequences, translation control sequences, origins of replication, and other regulatory sequences that are compatible with the recombinant microorganism and that control the expression of nucleic acid molecules of the present invention. In particular, recombinant molecules of the present invention at least include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant microorganisms of the present invention. A variety of such transcription control sequences are known to those skilled in the art; examples included, but are not limited to, tac, lac, trp, trc, oxy-pro, omp/lpp, rrnB, bacteriophage lambda (such as lambda P_{L}, also referred to herein as lambda PL) and lambda P_{R} (also referred to herein as lambda PR) and fusions that include such promoters), bacteriophage T7, T7lac, bacteriophage T3, bacteriophage SP6, SP01, alpha-mating factor, Pichia alcohol oxidase (AOX), antibiotic resistance gene, as other sequences capable of controlling gene expression in E. coli or methyltrophic yeast microorganisms; it is to be noted that this list is not intended to be limiting as many additional transcriptional control sequences are known. A particularly preferred recombinant molecule includes a nucleic acid molecule that encodes a mite Group 1 protein, operatively linked to the alcohol oxidase promoter AOX1. Another particularly preferred recombinant molecule includes a nucleic acid molecule that encodes a mite Group 1 protein operatively linked to the lambda PL promoter or the lambda PR promoter.

Recombinant molecules of the present invention can contain secretory signals (i.e., signal segment nucleic acid sequences) to enable an expressed fusion protein of the present invention to be secreted from the cell that produces the protein. Examples of suitable signal segments include any signal segment capable of directing the secretion of a protein of the present invention. Preferred signal segment sequences include, but are not limited to, mite Group 1 protein natural signal sequences and yeast alpha signal sequences, with the S. cerevisiae alpha signal sequence being particularly preferred for expression of a pro-form of a mite Group 1 protein in a methyltrophic yeast microorganism.

Another embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulation of mite Group 1 nucleic acid molecules of the present invention.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

One embodiment of a mite Group 1 protein of the present invention is a fusion protein. Suitable fusion segments for use with the present invention include, but are not limited to, segments that can: link two or more mite Group 1 proteins of the present invention to form multimers; enhance a protein's stability; facilitate the purification of a mite Group 1 protein; and/or to affect the immune response to a mite Group 1 protein. A suitable fusion segment can be a domain of any size that has the desired function (e.g., imparts increased stability, imparts increased immunogenicity to a protein, and/or simplifies purification of a protein). Fusion segments can be joined to amino and/or carboxyl termini of the mite Group 1 protein and can be susceptible to cleavage in order to enable straight-forward recovery of a mite Group 1 protein. Fusion proteins are preferably produced by culturing a recombinant cell transformed with a fusion nucleic acid molecule that encodes a protein including the fusion segment attached to either the carboxyl and/or amino terminal end of a mite Group 1 protein. Preferred fusion segments include a metal binding domain (e.g., a poly-histidine segment); an immunoglobulin binding domain (e.g., Protein A; Protein G; T cell; B cell; Fc receptor or complement protein antibody-binding domains); a sugar binding domain (e.g., a maltose binding domain); and/or a “tag” domain (e.g., at least a portion of β-galactosidase, a strep tag peptide, a T7 tag peptide, a Flag™ peptide, or other domains that can be purified using compounds that bind to the domain, such as monoclonal antibodies). More preferred fusion segments include metal binding domains, such as a poly-histidine segment; a maltose binding domain; a strep tag peptide, such as that available from Biometra in Tampa, Fla.; and an S10 peptide.

Effective culturing conditions to produce a recombinant mite Group 1 protein of the present invention include, but are not limited to, effective media, bioreactor, temperature, pH and oxygen conditions that permit protein production. An effective medium refers to any medium in which a cell is cultured to produce a mite Group 1 protein of the present invention. Such a medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for methyltrophic yeast or E. coli. Determining such culturing conditions are within the expertise of one of ordinary skill in the art.

Depending on the vector and host system used for production, resultant proteins of the present invention may either remain within the recombinant cell; be secreted into the culturing, or fermentation, medium; or be secreted into a space between two cellular membranes. In accordance with the present invention, recombinant mite Group 1 proteins produced by a methyltrophic yeast microorganism of the present invention are preferably secreted into the culturing medium, and recombinant mite Group 1 proteins produced by E. coli form inclusion bodies within the E. coli microorganism. As used herein, recovering a protein from a methyltrophic yeast microorganism refers to collecting the medium containing the yeast and the protein and need not imply additional steps of separation or purification. In a preferred embodiment, the protein is in the medium and, hence, can be easily separated from the yeast. Also, as used herein, the phrases isolating an inclusion body from an E. coli microorganism or recovering protein from the inclusion bodies do not imply any specified degree of separation or purification.

Proteins of the present invention can be purified using a variety of purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins of the present invention are preferably retrieved in “substantially pure” form. As used herein, “substantially pure” refers to a purity that allows for the effective use of the protein as a diagnostic, therapeutic or prophylactic.

A preferred method to purify a mite Group 1 protein from a mite Group 1 transformed-methyltrophic yeast microorganism is to recover the medium in which the mite Group 1 transformed methyltrophic yeast microorganism was cultured, and then to purify the mite Group 1 protein using conventional chromatography techniques. Preferred is a method by which the culturing medium is subjected to ion-exchange chromatography. Preferred ion exchange resins to use include cationic ion-exchange resins, particularly SP-Sepharose (available from Amersham-Pharmacia Biotech, Piscataway, N.J.) at about pH 4.5.

A preferred method to produce a recombinant mite Group 1 protein in E. coli is to culture a transformed E. coli microorganism of the present invention under conditions that cause the mite Group 1 protein to form an inclusion body within the microorganism. Such conditions are known in the art. An inclusion body, as used herein, is a highly aggregated, insoluble form of a mite Group 1 protein present in the E. coli microorganism. These inclusion bodies are thought to contain mis-folded (i.e., improperly folded) denatured protein. Mite Group 1 protein inclusion bodies are then recovered by lysing the E. coli microorganisms. Methods to recover inclusion bodies and purify and refold E. coli expressed proteins are known in the art, see, for example, Deutscher, ed., 1990, Guide to Protein Purification Academic Press, San Diego, Calif., which is incorporated by reference herein in its entirety. Methods to lyse E. coli are known in the art, and include methods such as enzymatic lysis, mechanical lysis, and liquid shear lysis; see, for example, Deutscher, ibid. A preferred method to lyse transformed E. coli is by mechanical means, most preferably with a microfluidizer. Insoluble proteins can be recovered by centrifugation. To solubilize the insoluble proteins, a variety of reagents can be used. Suitable reagents include: guanidine-hydrochloride (HCl), preferably at a pH from about pH 7 to about pH 8 and at a concentration of from about 5 molar (M) to about 8 M; urea, preferably at a concentration of from about 6 M to about 8 M; sodium dodecyl sulfate; alkaline pH (greater than pH 9); and/or acetonitrile/propanol. Preferred methods to solubilize include use of 8 M urea in Tris buffer, pH 9.5, in 100 millimolar (mM) β-mercaptoethanol. Solubilized proteins can be refolded directly or subjected to additional purification step(s). A preferred method is to purify the mite Group 1 protein further before refolding. Any number of different types of resins suitable for protein purification may be used; preferred is an anion-exchange type resin such as, for example, Q-SEPHAROSE™ resin (available from Amersham-Pharmacia Biotech). To refold the solubilized protein directly or after purification, a number of methods are known in the art; see, for example, Deutscher, M. (1990), ibid. The term, refold, as used herein, refers to conditions in which reduced proteins can revert to their correct conformations, including restoring the correct disulfide bridges. Preferred methods to refold include (a) using glutathione to form mixed disulfides and/or (b) using high pH. For method (a), steps involved include: (i) reduction of the protein with a reducing agent, such as dithiothreitol, preferably 6 mM dithiothreitol for at least about 30 minutes; (ii) addition of oxidized glutathione, preferably at a concentration of from about 25 mM to about 100 mM, with about 25 mM being particularly preferred; (iii) dilution of the mixture to a urea concentration of preferably between about 0.5 M and 1 M, with 0.75 M urea being particularly preferred (assuming solubilization is conducted in the presence of urea) using a buffer, preferably 50 mM Tris, pH 9.5, with the addition of from about 5 mM to about 25 mM, preferably 6.5 mM, cysteine or reduced glutathione; (iv) incubation for about 10 to about 20 hours at 4° C.; and (v) dialysis against a buffer to remove urea; preferred buffers are phosphate buffered saline (PBS) at about pH 7.5 or 50 mM Tris-HCl, pH 7.5. For method (b), steps involved include: (i) adjusting pH of the solubilized protein to about pH 10; (ii) reducing the protein by treatment with 100 mM β-mercaptoethanol; and (iii) refolding by dialysis against PBS, pH 7.2 or 50 mM Tris-HCl, pH 7.5.

One embodiment of the present invention is a composition that, when administered to an animal in an effective manner, is capable of reducing an allergic response to a mite Group 1 protein in a mite Group 1 protein allergic animal. Such a composition can function as a preventative, or prophylactic, or as a therapeutic, or treatment. Such a composition includes an isolated mite Group 1 variant protein of the present invention and at least one of the following components: an excipient, an adjuvant, and a carrier that the animal can tolerate. Examples of excipients, adjuvants and carriers are found throughout the art; see, for example, U.S. Pat. No. 5,958,880, ibid. and 5,840,695, ibid.

A mite Group 1 variant protein of the present invention is genetically engineered to lessen or completely abolish a mite Group 1 protein's ability to bind to IgE. Such a molecule can be used to reduce an animal's allergic response to exposure to a mite Group 1 protein.

Suitable protocols by which to administer compositions of the present invention in an effective manner can vary according to individual dose size, number of doses, frequency of dose administration, and mode of administration. Determination of such protocols can be accomplished by those skilled in the art. An effective dose refers to a dose capable of treating an animal against hypersensitivity to mite allergens. Effective doses can vary depending upon, for example, the composition used and the size and type of the recipient animal, i.e. what species. Effective doses to immunomodulate an animal against a mite Group 1 protein include doses administered over time that are capable of alleviating a hypersensitive response by an animal to a mite Group 1 protein. For example, a first tolerizing dose can comprise an amount of a composition of the present invention that causes a minimal hypersensitive response when administered to a hypersensitive animal. A second tolerizing dose can comprise a greater amount of the same composition than the first dose. Effective tolerizing doses can comprise increasing concentrations of the composition necessary to tolerize an animal such that the animal does not have a hypersensitive response to exposure to a mite Group 1 protein. An effective dose to desensitize an animal can comprise a concentration of a composition of the present invention sufficient to block an animal from having a hypersensitive response to exposure to a mite allergen present in the environment of the animal. Effective desensitizing doses can include repeated doses having concentrations of a composition that cause a minimal hypersensitive response when administered to a hypersensitive animal.

A suitable single dose is a dose that is capable of treating an animal against hypersensitivity to a mite Group 1 protein when administered one or more times over a suitable time period. For example, a preferred single dose of a mite Group 1 protein-containing composition is from about 0.5 nanograms (ng) to about 1 gram (g) of the protein per kilogram body weight of the animal. Further treatments with the composition can be administered from about 1 day to 1 year after the original administration. Further treatments with the composition preferably are administered when the animal is no longer protected from hypersensitive responses to mite Group 1 proteins. Particular administration doses and schedules can be developed by one of the skill in the art based upon the parameters discussed above. Modes of administration can include, but are not limited to, subcutaneous, intradermal, intravenous, nasal, oral, transdermal and intramuscular routes.

A composition of the present invention can be used in conjunction with other compounds capable of modifying an animal's hypersensitivity to mite allergens. For example, an animal can be treated with compounds capable of modifying the function of a cell involved in a hypersensitive response, compounds that reduce allergic reactions, such as by systemic agents or anti-inflammatory reagents (e.g., anti-histamines, anti-steroid reagents, anti-inflammatory reagents and reagents that drive immunoglobulin heavy chain class switching from IgE to IgG). Suitable compounds useful for modifying the function of a cell involved in a hypersensitive response include, but are not limited to, antihistamines, cromolyn sodium, theophylline, cyclosporin A, adrenalin, cortisone, compounds capable of regulating cellular signal transduction, compounds capable of regulating adenosine 3′, 5′ cyclic phosphate (cAMP) activity, and compounds that block IgE activity, such as peptides from IgE or IgE specific Fc receptors, antibodies specific for peptides from IgE or IgE-specific Fc receptors, or antibodies capable of blocking binding of IgE to Fc receptors.

A composition of the present invention can also be used in conjunction with other antigens to prevent or treat allergic diseases. Examples of antigens causing allergy include, but are not limited to those disclosed in U.S. Pat. No. 5,945,294, ibid.; U.S. Pat. No. 5,958,880, ibid.; WO 98/45707, ibid.; WO 99/38974, ibid.; and U.S. Ser. No. 09/287,380, ibid.

Additional teachings with respect to compositions and uses thereof to reduce allergy can be found, for example, in U.S. Pat. No. 5,958,880, ibid. and 5,840,695, ibid.

The following examples are provided for the purposes of illustration and are not intended to limit the scope of the present invention. The following examples include a number of recombinant DNA and protein chemistry techniques known to those skilled in the art; see, for example, Sambrook et al., ibid.

### EXAMPLES

### Example 1

This Example describes certain novel mite Group 1 nucleic acid molecules of the present invention. These mutant genes have substitutions and/or deletions that result in proteins with altered conformation and biological properties. The mutant genes are created by PCR mutagenesis of the clones previously isolated and optimized for expression, as described in related PCT publication WO 01/29078A2, which is herein incorporated by reference.

A. This example describes Der p 1 Group 1 mutant cDNA nucleic acid molecule, denoted herein as nDerp1₈₉₄. The coding strand of nDerp1₈₉₄, represented by SEQ ID NO:1, incorporates Pichia-preferred codon changes and has a deletion of 12 base pairs. The reverse complement of SEQ ID NO:1 is SEQ ID NO:3. Nucleic acid molecule nDerp1₈₉₄ encodes a pro-form of a Der p1 Group 1 protein, namely PDerp1_{298:ΔC31-34}. Translation of the coding strand (SEQ ID NO:1) yields the protein represented by SEQ ID NO:2. The deletion of amino acids 31 through 34 should result in disruption of the C32-C72 disulfide bond, as well as destruction of the protease activity (at C34).

B. This example describes Der p 1 Group 1 mutant cDNA nucleic acid molecule, denoted herein as nDerp1₆₅₄, the coding strand of which, represented by SEQ ID NO:4, incorporates Pichia-preferred codon changes and has a deletion of 12 base pairs. The reverse complement of SEQ ID NO:4 is SEQ ID NO:6. Nucleic acid molecule nDerp1₆₅₄ encodes a mature form of a Der p 1 Group 1 protein, namely PDerp1_{218:ΔC31-34}. Translation of the coding strand (SEQ ID NO:4) yields the protein represented by SEQ ID NO:5. The deletion of amino acids 31 through 34 should result in disruption of the C32-C72 disulfide bond, as well as destruction of the protease activity (at C34).

C. This example describes Der p 1 Group 1 mutant cDNA nucleic acid molecule, denoted herein as nDerp1₉₀₆ₐ, the coding strand of which, represented by SEQ ID NO:7, incorporates Pichia-preferred codon changes and has a substitution to change the cysteine at position 4 of the mature protein to serine. The reverse complement of SEQ ID NO:7 is SEQ ID NO:9. Nucleic acid molecule nDerp1₉₀₆ₐ encodes a pro-form of a Der p 1 Group 1 protein, namely PDerp 1_{302:C4S}. Translation of the coding strand (SEQ ID NO:7) yields the protein represented by SEQ ID NO:8. The substitution of amino acid C4 should result in disruption of the C4-C118 disulfide bond.

D. This example describes Der p 1 Group 1 mutant cDNA nucleic acid molecule, denoted herein as nDerp1_{906b}, the coding strand of which, represented by SEQ ID NO:10, incorporates Pichia-preferred codon changes and has two substitutions to change the cysteines at positions 4 and 31 to serines. The reverse complement of SEQ ID NO:10 is SEQ ID NO:12. Nucleic acid molecule nDerp1_{906b} encodes a pro-form of a Der p 1 Group 1 protein, namely PDerp1_{302:C4S,C31S}. Translation of the coding strand (SEQ ID NO:10) yields the protein represented by SEQ ID NO:11. The substitution of amino acids 4 and 31 should result in disruption of the C4-C118 disulfide bond and the C31-C71 disulfide bond.

E. This example describes Der p 1 Group 1 mutant cDNA nucleic acid molecule, denoted herein as nDerp1_{906c}, the coding strand of which, represented by SEQ ID NO:13, incorporates Pichia-preferred codon changes and has three substitutions to change the cysteines at positions 4, 31 and 71 to serines. The reverse complement of SEQ ID NO:13 is SEQ ID NO:15. Nucleic acid molecule nDerp1_{906c} encodes a pro-form of a Der p 1 Group 1 protein, namely PDerp1_{302:C4S,C31S,C71S}. Translation of the coding strand (SEQ ID NO:13) yields the protein represented by SEQ ID NO:14. The substitution of amino acids 4, 31 and 71 should result in disruption of the C4-C117 and the C32-C72 disulfide bond.

F. This example describes Der p 1 Group 1 mutant cDNA nucleic acid molecule, denoted herein as nDerp1₈₈₈, the coding strand of which, represented by SEQ ID NO:16, incorporates Pichia-preferred codon changes and has a deletion of 18 base pairs. The reverse complement of SEQ ID NO:16 is SEQ ID NO:18. Nucleic acid molecule nDerp1₈₈₈ encodes a pro-form of a Der p 1 Group 1 protein, namely PDerp1_{296:ΔR151-156}. Translation of the coding strand (SEQ ID NO:16) yields the protein represented by SEQ ID NO:17. The deletion of amino acids 151 through 156 should result in disruption of a surface exposed IgE epitope, but is believed to be outside major T cell epitopes.

G. This example describes Der p 1 Group 1 mutant cDNA nucleic acid molecule, denoted herein as nDerp1₉₀₉ₐ, the coding strand of which, represented by SEQ ID NO:19, incorporates E. coli-preferred codon changes and has two substitutions to change the cysteines at positions 4 and 117 to serines. The reverse complement of SEQ ID NO:19 is SEQ ID NO:21. Nucleic acid molecule nDerp1₉₀₉ encodes a pro-form of a Der p 1 Group 1 protein, namely PDerp1_{303:C4S,C117S}. Translation of the coding strand (SEQ ID NO:19) yields the protein represented by SEQ ID NO:20. The substitution of amino acids C4 and C117 should result in disruption of the C4-C 117 disulfide bond.

H. This example describes Der p 1 Group 1 mutant cDNA nucleic acid molecule, denoted herein as nDerp1₈₉₇ₐ, the coding strand of which, represented by SEQ ID NO:22, incorporates E. coli-preferred codon changes and has a deletion of 12 base pairs. The reverse complement of SEQ ID NO:22 is SEQ ID NO:24. Nucleic acid molecule nDerp1₈₉₇ₐ, encodes a pro-form of a Der p 1 Group 1 protein, namely PDerp1_{229:ΔC31-34}. Translation of the coding strand (SEQ ID NO:22) yields the protein represented by SEQ ID NO:23. The deletion of amino acids 31 through 34 should result in disruption of the C32-C72 disulfide bond, as well as destruction of the protease activity (at C34).

I. This example describes Der p 1 Group 1 mutant cDNA nucleic acid molecule, denoted herein as nDerp1_{897b}, the coding strand of which, represented by SEQ ID NO:25, incorporates E. coli-preferred codon changes, has a deletion of 12 base pairs, and a substitution to change the cysteine at position 71 to serine. The reverse complement of SEQ ID NO:25 is SEQ ID NO:27. Nucleic acid molecule nDerp1₈₉₇ₐ encodes a pro-form of a Der p 1 Group 1 protein, namely PDerp1_{229:ΔC31-34,C71S}. Translation of the coding strand (SEQ ID NO:25) yields the protein represented by SEQ ID NO:26. The deletion of amino acids 31 through 34 should result in disruption of the C32-C72 disulfide bond, and the protease activity (of C34), and the substitution of C71 should result in the destruction of the C32-C71 bond.

J. This example describes Der p 1 Group 1 mutant cDNA nucleic acid molecule, denoted herein as nDerp1₈₉₁, the coding strand of which, represented by SEQ ID NO:28, incorporates E. coli-preferred codon changes and has a deletion of 18 base pairs. The reverse complement of SEQ ID NO:28 is SEQ ID NO:30. Nucleic acid molecule nDerp1₈₉₁, encodes a pro-form of a Der p 1 Group 1 protein, namely PDerp1_{227:ΔR151-R156}. Translation of the coding strand (SEQ ID NO:28) yields the protein represented by SEQ ID NO:29. The deletion of amino acids 151 through 156 should result in disruption of a surface exposed IgE epitope, but is believed to be outside major T cell epitopes.

K. This example describes Der p 1 Group 1 mutant cDNA nucleic acid molecule, denoted herein as nDerp1_{909b}, the coding strand of which, represented by SEQ ID NO:31, incorporates E. coli-preferred codon changes and has one substitution to change the cysteine at position 4 to serine. The reverse complement of SEQ ID NO: 31 is SEQ ID NO:33. Nucleic acid molecule nDerp1_{909b} encodes a pro-form of a Der p 1 Group 1 protein, namely PDerp1_{303:C4S}. Translation of the coding strand (SEQ ID NO:31) yields the protein represented by SEQ ID NO:32. The substitution of amino acid C4 should result in disruption of the C4-C117 disulfide bond.

L. This example describes Der p 1 Group 1 mutant cDNA nucleic acid molecule, denoted herein as nDerp1_{909c}, the coding strand of which, represented by SEQ ID NO:34, incorporates E. coli-preferred codon changes and has two substitutions to change the cysteines at positions 4 and 31 to serines. The reverse complement of SEQ ID NO:34 is SEQ ID NO:36. Nucleic acid molecule nDerp1_{909c} encodes a pro-form of a Der p 1 Group 1 protein, namely PDerp1_{303:C4S,C31S}. Translation of the coding strand (SEQ ID NO:34) yields the protein represented by SEQ ID NO:35. The substitution of amino acids C4 and C31 should result in disruption of the C4-C117 disulfide bond and the C31-C71 disulfide bond.

M. This example describes Der p 1 Group 1 mutant cDNA nucleic acid molecule, denoted herein as nDerp1_{909d}, the coding strand of which, represented by SEQ ID NO:37, incorporates Ecoli-preferred codon changes and has two substitutions to change the cysteines at positions 4 and 71 to serines. The reverse complement of SEQ ID NO:37 is SEQ ID NO:39. Nucleic acid molecule nDerp1_{909d} encodes a pro-form of a Der p 1 Group 1 protein, namely PDerp1_{303:C4S,C71S}. Translation of the coding strand (SEQ ID NO:37) yields the protein represented by SEQ ID NO:38. The substitution of amino acids C4 and C71 should result in disruption of the C4-C117 disulfide bond and the C31-C71 disulfide bond.

N. This example describes Der p 1 Group 1 mutant cDNA nucleic acid molecule, denoted herein as nDerp1₉₀₉ₑ, the coding strand of which, represented by SEQ ID NO:40, incorporates E. coli-preferred codon changes and has three substitutions to change the cysteines at positions 4, 31, and 71 to serines. The reverse complement of SEQ ID NO:40 is SEQ ID NO:42. Nucleic acid molecule nDerp1₉₀₉ₑ encodes a pro-form of a Der p 1 Group 1 protein, namely PDerp1_{303:C4S, C31S,C71S}. Translation of the coding strand (SEQ ID NO:40) yields the protein represented by SEQ ID NO:41. The substitution of amino acids C4, C31 and C71 should result in disruption of the C4-C117 disulfide bond and the C31-C71 disulfide bond.

### Example 2

This Example describes the expression and purification of the variant mite Group 1 proteins of the present invention from supernatant cultures of recombinant Pichia microorganisms.

Recombinant P. pastoris microorganisms were routinely cultured on YPD culture medium (1% yeast extract, 2% peptone, 2% dextrose). His+ transformants were selected on MD culture medium (1.34% yeast nitrogen base, 0.00004% biotin, 2% dextrose). Small-scale inductions of expression of recombinant P. pastoris strains containing the nucleic acid molecules of the present invention were performed using BMG or BMM culture media which were composed of the following: 100 mM potassium phosphate, pH6.0, 1.34% yeast nitrogen base, 0.00005% biotin and either 1% glycerol (BMG) or 0.5% methanol (BMM). For each recombinant strain grown and induced, a single colony of that strain was inoculated into 25 ml BMG culture medium in a 250 ml baffled flask covered with a porous silicon rubber stopper to allow maximum aeration. The culture was grown at 28° C. with shaking to an optical density (A600) of about 1.0. The culture was then pelleted for 10 min at 3000×g (times gravity) and resuspended in 250 ml BMM culture medium at an optical density (A600) of about 1.0 in a 2-liter (L) baffled flask with a porous silicon rubber stopper to induce expression of a Der f 1 nucleic acid molecule operatively linked to the AOX promoter. The culture was incubated at 28° C. for 4 days; methanol was added daily to a final concentration of 0.5% volume/volume (v/v). The entire culture volume was concentrated to 20% of original volume by tangential flow filtration (3000 MW cutoff, available from AG Technologies Needham, Mass.) and analyzed by SDS-PAGE.

The supernatant from the culture was recovered and diluted 1:3 (v/v) with 25 mM sodium acetate pH 4.5 (Buffer A) and loaded onto a 1.6×10 mm SP-Sepharose (available from Amersham-Pharmacia Biotech, Piscataway, N.J.) previously equilibrated with 25 mM sodium acetate, pH 4.5. Bound protein was eluted with a linear salt gradient to 100% Buffer B (25 mM sodium acetate, 1 M NaCl, pH 4.5) in 20 to 25 column volumes. Fractions (5.0 ml) were collected and analyzed by SDS-PAGE and reverse phase RPC18 chromatography. Recombinant proteins produced by recombinant P. pastoris microorganisms eluted at 0.1 to 0.15 M NaCl and migrated as a diffuse band with an apparent molecular weight ranging from about 40 to 46 kD. Fractions containing Der f 1 proteins (>90% homogeneous) were pooled and concentrated using a 10-kD molecular weight cut-off (MWCO) centriprep concentrator.

### Example 3

This example discloses a procedure for the removal of endotoxin from inclusion bodies containing Dermatophagoides pteronyssinus (Der p) group I allergens.

Cells expressing recombinant Der p group I allergens were grown using standard protocols. Pelleted cells were resuspended in TEP buffer (100 mM Tris-HCL, pH 8.5, 10 mM EDTA, 1 mM PMSF) (10 ml/gram of cells), homogenized twice, 30 seconds each, and then microfluidized for 50 pulses. The cell homogenate was centrifuged at 1000-2000×g for 20 minutes at 4° C. and the supernatant discarded. The pellet was resuspended in TEP buffer (10 ml/gram of cells), homogenized for 30 seconds and the homogenate centrifuged at 1000-2000×g for 20 minutes at 4° C. The supernatant was discarded and the pellet resuspended in TEP buffer (10 ml/gram original cell pellet) containing 0.5%(w/v) deoxycholate. After homogenizing for 30 seconds, the sample was mixed on an end-over-end rotator for 30 minutes at 4° C. The homogenate was centrifuged at 1000-2000×g for 20 minutes at 4° C., the supernatant discarded and the pellet resuspended in TEP buffer (10 ml/gram original cell pellet) containing 0.5% deoxycholate. The pellet was homogenized for 30 seconds followed by mixing on an end-over-end rotator for 30 minutes at 4° C. and the homogenate centrifuged at 1000-2000×g for 20 minutes at 4° C. The supernatant was discarded and the pellet resuspended in TEP buffer (10 ml/gm original cell pellet) and homogenized for 30 seconds. The homogenate was centrifuged at 1000-2000×g for 20 minutes at 4° C. and the supernatant discarded. The pellet was resuspended in TEP buffer containing 8M urea and 1% (w/v) DTT and homogenized for 30 seconds. (The volume of buffer can be adjusted depending on the desired number of sizing column runs. Each run can take a 50 ml load on a 5 cm×100 cm column of Sephacryl S-200.) The homogenate was mixed on an end-to-end rotor for 30 minutes at room temperature (RT), the mixture centrifuged at 30,000×g for 20 minutes at 12° C. and the supernatant removed. The supernatant is referred to as S6 and is retained for assay and further processing.

### Example 4

This example illustrates the method used to further purify and refold the group I dust mite allergens present in the S6 fraction of Example 3. All materials used in this method must be free of endotoxin. The size exclusion column (SEC) used was stored in 25% ethanol to prevent endotoxin contamination of the column. Prior to use, the ethanol was removed with water, the column washed with 4M urea in 25 mM Tris, pH 9.5 and then equilibrated with the appropriately buffered solution.

Prior to loading the sample on the column, the protein concentration of the S6 fraction was determined. In general, there is a balance between the pre-column protein concentration, monomer yield and total recovery. The higher the concentration of the load, the greater the aggregate formation and the less the monomer yield. However, the total monomer recovery was also factored into determining the ideal concentration of the load. Some muteins (in particular proDerp1C4SC117s and proDerp1C4SC31S) have a tendency to fall out of solution above a certain concentration. (>0.05-0.06 mg/ml) in PBS and care must be taken when concentrating these muteins in preparation for the SEC chromatography and during concentration of the final monomer product.

A 5 cm×100 cm (1.96 L bed volume (bv)) Sephacryl S-200 column was used to fractionate the muteins in the S6 fraction of Example 1. The column was washed as described above and equilibrated with 20 mM Tris-Cl, pH8.6 at a flow rate of 10 ml/min. A 50 ml load of fraction S6 was loaded onto the column using a superloop at 10 ml/min and 14 ml fractions were collected over approximately one column volume. Fractions corresponding to the mutein dimer peak were pooled and dialyzed overnight at 4° C. against Takahashi PBS (137 mM NaCl, 2.6 mM KCL, 10 mM phosphate, pH 7.4). (The identities of the dimer and monomer peaks were determined by comparing the elution peak times against the elution times of known size standards run over the same column). The dialyzed sample was then concentrated using a stirred-cell concentrator, the protein concentration determined and the sample re-applied to the Sephacryl S-100 column which had been equilibrated in Takahashi PBS. Takahashi PBS is also used as the running buffer for the second run. As before, 14 ml fractions were collected over one column volume and the flow rate was 10 ml/min. Fractions corresponding to the monomer peak were pooled, the protein concentration determined and the sample concentrated to approximately 0.2 mg/ml or in the case of proDer p1C4SC117S and proDer p1 C4Sc31S to approximately 0.06 mg/ml.

### Example 5

This example describes the evaluation of the IgE binding ability of the wt and mutated allergens (muteins) purified and refolded as described in Examples 3 and 4.

A. Procedure for Antigen Coating of Micro-Titer Wells.

A 10 ml stock solution of each allergen to be evaluated was prepared at a concentration of 10 μg/ml in Bicarbonate Coating Buffer (50 mM bicarbonate, pH 9.5). For each allergen stock solution, seven serial, 2-fold dilutions were performed so that the concentration of allergen in the final tube was 0.078 μg/ml. The wells of a microtiter plate were coated with individual antigens by adding 100 μl of stock or diluted allergen to individual wells and incubating the plate for 16-18 hours at 4-8° C. All wells in a horizontal row were coated using the same allergen at the same dilution (see Figure 1 below). After incubation, the plate was washed twice with PBS-Tween (PBS, 0.5% Tween-20) and then 450 μl of 1% Monoethanolamine Blocking Solution (1% monoethanolamine (w/v) in water) were added to each well and the plate incubated at room temperature (RT) for at least 1 hour. The plate was then washed three times with PBS-Tween, blotted with a paper towel and dried by incubation in a plate dryer for approximately two hours. The plates were stored in a sealed container at 4-8° C. until needed.

B. Procedure for Evaluating IgE Binding Activity of Microtiter-Well Bound Allergens

The ability of the well-bound allergens to bind IgE from allergic individuals was tested by performing an ELISA using pooled human sera collected from people known to be allergic to Dermatophagoides pteronyssinus. Pooled non-allergic, human sera was used as a negative control. To determine the background ELISA levels, the same ELISA protocol was followed but Tris-HCl Diluent (50 mM Tris-HCl, pH7.5, 2 mM MgCl₂, 145 mM NaCl, 1% BSA, 0.3 mM NaN₃, 0.05% Tween-20) was added to the wells instead of sera.

The ELISA was performed as follows: Negative control sera (Negative) was diluted 1:30 with Tris-HCl Diluent. High titer positive control sera (Positive) was diluted 1:30, 1:120 and 1:480 with Tris-HCl Diluent. 100 μl of either positive sera, negative sera or diluent were added to the micro-titer plate as shown in Figure 1.

FIG. 1. Layout of Allergens and Sera in Micro-titer Plate

| | Antigen | | | | | | | | | | | |
|---|---|---|---|---|---|---|---|---|---|---|---|---|
| Row | (μg/ml) | 1 | 2 | 3 | 4 | 5 | 6 | 7 | 8 | 9 | 10 | 11 12 |

| | | | | | | |
|---|---|---|---|---|---|---|
| A | 10 | Positive | Positive | Positive | Negative | Dilu- |
| | | 1:30 | 1:120 | 1:480 | 1:30 | ent |
| B | 5 | Positive | Positive | Positive | Negative | Dilu- |
| | | 1:30 | 1:120 | 1:480 | 1:30 | ent |
| C | 2.5 | Positive | Positive | Positive | Negative | Dilu- |
| | | 1:30 | 1:120 | 1:480 | 1:30 | ent |
| D | 1.25 | Positive | Positive | Positive | Negative | Dilu- |
| | | 1:30 | 1:120 | 1:480 | 1:30 | ent |
| E | 0.625 | Positive | Positive | Positive | Negative | Dilu- |
| | | 1:30 | 1:120 | 1:480 | 1:30 | ent |
| F | 0.313 | Positive | Positive | Positive | Negative | Dilu- |
| | | 1:30 | 1:120 | 1:480 | 1:30 | ent |
| G | 0.156 | Positive | Positive | Positive | Negative | Dilu- |
| | | 1:30 | 1:120 | 1:480 | 1:30 | ent |
| H | 0.078 | Positive | Positive | Positive | Negative | Dilu- |
| | | 1:30 | 1:120 | 1:480 | 1:30 | ent |

The plate was incubated for 16-18 hours at 4-8° C. after which the wells were washed once with Wash Buffer (50 mM Tris, pH 7.5, 145 mM NaCl, 0.3 mM NaN₃, 0.05% Tween-20). 100 μl of biotinylated human Fc receptor (10 ng/ml in Tris-HCl Diluent) were then added to each well and the plate incubated at RT for 2 hours. The wells were washed with Wash Buffer and 100 μl of streptavidin-alkaline phosphatase conjugate (125 ng/ml Tris-HCl Diluent) were added to each well and the plate incubated -1 hour at RT. The wells were washed four times with Wash Buffer, the plate blotted dry and 100 μl p-Nitrophenyl Phosphate Substrate Solution (Moss, Inc.) were added to each well. Following incubation of the plate at RT for 1 hour, the development reaction was stopped by the addition of 50 μl of 20 mM cysteine to each well. The optical density of each well was measured at 405 nm and the results are shown in Table 1. The values shown are the averages of samples run in duplicate or triplicate.

TABLE 1
ELISA Results Using wt Der p1 and Der p1 Muteins
Der p1HCY462-recombinant wt Der p1 expressed in yeast - lot#1

| | Positive Sera Pool | | | Negative | |
|---|---|---|---|---|---|
| ug/mL | 1:31 | 1:121 | 1:481 | 1:31 | Background |
| 10 | 2.209 | 0.898 | 0.351 | 0.112 | 0.103 |
| 5 | 2.179 | 0.823 | 0.341 | 0.105 | 0.105 |
| 2.5 | 2.096 | 0.788 | 0.309 | 0.100 | 0.101 |
| 1.25 | 1.963 | 0.733 | 0.303 | 0.099 | 0.099 |
| 0.625 | 1.761 | 0.706 | 0.292 | 0.113 | 0.106 |
| 0.3125 | 1.364 | 0.449 | 0.255 | 0.119 | 0.110 |
| 0.15625 | 1.041 | 0.445 | 0.225 | 0.121 | 0.121 |
| 0.078125 | 0.704 | 0.342 | 0.195 | 0.126 | 0.110 |

Der p1HCY462-recombinant wt Der p1 expressed in yeast - lot#2

| | Positive Sera Pool | | | Negative | |
|---|---|---|---|---|---|
| ug/mL | 1:31 | 1:121 | 1:481 | 1:31 | Background |
| 10 | 2.409 | 0.972 | 0.401 | 0.128 | 0.135 |
| 5 | 2.420 | 0.951 | 0.374 | 0.094 | 0.103 |
| 2.5 | 2.430 | 0.918 | 0.356 | 0.097 | 0.111 |
| 1.25 | 2.195 | 0.845 | 0.336 | 0.106 | 0.107 |
| 0.625 | 1.689 | 0.725 | 0.300 | 0.105 | 0.108 |
| 0.3125 | 1.318 | 0.429 | 0.243 | 0.113 | 0.105 |
| 0.15625 | 0.800 | 0.409 | 0.208 | 0.117 | 0.111 |
| 0.078125 | 0.519 | 0.294 | 0.170 | 0.128 | 0.106 |

Der p1HCY484-recombinant wt Der p1 expressed in yeast - lot #1

| | Positive Sera Pool | | | Negative | |
|---|---|---|---|---|---|
| ug/mL | 1:31 | 1:121 | 1:481 | 1:31 | Background |
| 10 | 2.289 | 0.967 | 0.406 | 0.104 | 0.117 |
| 5 | 1.869 | 0.840 | 0.365 | 0.105 | 0.109 |
| 2.5 | 1.725 | 0.762 | 0.343 | 0.106 | 0.105 |
| 1.25 | 1.514 | 0.678 | 0.305 | 0.104 | 0.111 |
| 0.625 | 1.264 | 0.596 | 0.277 | 0.105 | 0.111 |
| 0.3125 | 0.980 | 0.399 | 0.238 | 0.106 | 0.111 |
| 0.15625 | 0.776 | 0.411 | 0.213 | 0.112 | 0.116 |
| 0.078125 | 0.522 | 0.315 | 0.190 | 0.133 | 0.119 |

Der p1HCY484-recombinant wt Der p1 expressed in yeast - lot #2

| | Positive Sera Pool | | | Negative | |
|---|---|---|---|---|---|
| ug/mL | 1:31 | 1:121 | 1:481 | 1:31 | Background |
| 10 | 1.071 | 0.529 | 0.265 | 0.129 | 0.105 |
| 5 | 1.091 | 0.504 | 0.240 | 0.107 | 0.101 |
| 2.5 | 1.194 | 0.562 | 0.248 | 0.122 | 0.101 |
| 1.25 | 1.112 | 0.520 | 0.246 | 0.099 | 0.106 |
| 0.625 | 0.887 | 0.446 | 0.223 | 0.108 | 0.106 |
| 0.3125 | 0.639 | 0.289 | 0.198 | 0.109 | 0.115 |
| 0.15625 | 0.471 | 0.283 | 0.174 | 0.117 | 0.123 |
| 0.078125 | 0.360 | 0.222 | 0.149 | 0.120 | 0.100 |

Native Der p1

| | Positive Sera Pool | | | Negative | |
|---|---|---|---|---|---|
| ug/mL | 1:31 | 1:121 | 1:481 | 1:31 | Background |
| 10 | 4.200 | 2.364 | 0.899 | 0.122 | 0.115 |
| 5 | 4.087 | 1.954 | 0.792 | 0.115 | 0.124 |
| 2.5 | 3.942 | 1.615 | 0.715 | 0.107 | 0.143 |
| 1.25 | 3.588 | 1.537 | 0.568 | 0.107 | 0.109 |
| 0.625 | 3.061 | 1.285 | 0.537 | 0.113 | 0.110 |
| 0.3125 | 2.250 | 0.717 | 0.423 | 0.106 | 0.105 |
| 0.15625 | 1.488 | 0.665 | 0.279 | 0.118 | 0.116 |
| 0.078125 | 0.941 | 0.465 | 0.238 | 0.121 | 0.119 |

Der p1HCE389-recombinant wt proDer p1 expressed in E. coli

| | Positive Sera Pool | | | Negative | |
|---|---|---|---|---|---|
| ug/mL | 1:31 | 1:121 | 1:481 | 1:31 | Background |
| 10 | 4.214 | 2.879 | 0.937 | 0.153 | 0.163 |
| 5 | 4.174 | 2.257 | 0.723 | 0.138 | 0.151 |
| 2.5 | 4.145 | 2.130 | 0.685 | 0.141 | 0.153 |
| 1.25 | 4.092 | 2.066 | 0.693 | 0.137 | 0.136 |
| 0.625 | 3.829 | 1.773 | 0.501 | 0.120 | 0.121 |
| 0.3125 | 3.043 | 1.177 | 0.363 | 0.108 | 0.100 |
| 0.15625 | 1.032 | 0.467 | 0.170 | 0.107 | 0.097 |
| 0.078125 | .694 | 0.340 | 0.150 | 0.128 | 0.105 |

Der p1HCE446-recombinant proDer p1 C4S C31S expressed in E. coli

| | Positive Sera Pool | | | Negative | |
|---|---|---|---|---|---|
| ug/mL | 1:31 | 1:121 | 1:481 | 1:31 | Background |
| 10 | 0.800 | 0.400 | 0.236 | 0.158 | 0.173 |
| 5 | 0.796 | 0.346 | 0.197 | 0.144 | 0.153 |
| 2.5 | 0.613 | 0.287 | 0.187 | 0.140 | 0.151 |
| 1.25 | 0.196 | 0.156 | 0.140 | 0.131 | 0.143 |
| 0.625 | 0.162 | 0.138 | 0.134 | 0.186 | 0.145 |
| 0.3125 | 0.158 | 0.135 | 0.135 | 0.125 | 0.141 |
| 0.15625 | 0.163 | 0.147 | 0.150 | 0.138 | 0.158 |
| 0.078125 | 0.174 | 0.159 | 0.164 | 0.170 | 0.163 |

Der p1HCE433-recombinant proDer
p1 C4S C117S expressed in E. coli

| | Positive Sera Pool | | | | |
|---|---|---|---|---|---|
| ug/mL | 1:31 | 1:121 | 1:481 | Negative | Background |
| 10 | 0.623 | 0.386 | 0.230 | 0.184 | 0.213 |
| 5 | 0.567 | 0.340 | 0.214 | 0.177 | 0.201 |
| 2.5 | 0.733 | 0.353 | 0.234 | 0.180 | 0.185 |
| 1.25 | 0.317 | 0.218 | 0.195 | 0.171 | 0.184 |
| 0.625 | 0.205 | 0.175 | 0.160 | 0.166 | 0.176 |
| 0.3125 | 0.164 | 0.156 | 0.158 | 0.169 | 0.184 |
| 0.15625 | 0.161 | 0.160 | 0.155 | 0.152 | 0.170 |
| 0.078125 | 0.190 | 0.178 | 0.173 | 0.156 | 0.154 |

Der p1HCE400-recombinant proDer
p1Ä31-34 expressed in E. coli

| | Positive Sera Pool | | | | |
|---|---|---|---|---|---|
| ug/mL | 1:31 | 1:121 | 1:481 | Negative | Background |
| 10 | 0.741 | 0.402 | 0.237 | 0.140 | 0.138 |
| 5 | 0.653 | 0.398 | 0.219 | 0.116 | 0.121 |
| 2.5 | 0.543 | 0.315 | 0.184 | 0.118 | 0.119 |
| 1.25 | 0.420 | 0.240 | 0.160 | 0.111 | 0.122 |
| 0.625 | 0.251 | 0.167 | 0.127 | 0.106 | 0.111 |
| 0.3125 | 0.171 | 0.138 | 0.116 | 0.108 | 0.110 |
| 0.15625 | 0.140 | 0.127 | 0.116 | 0.127 | 0.109 |
| 0.078125 | 0.132 | 0.132 | 0.127 | 0.129 | 0.113 |

Der p1HCE413-recombinant proDer
p1Ä31-34 C71S expressed in E. coli

| | Positive Sera Pool | | | | |
|---|---|---|---|---|---|
| ug/mL | 1:31 | 1:121 | 1:481 | Negative | Background |
| 10 | 0.743 | 0.406 | 0.240 | 0.143 | 0.150 |
| 5 | 0.706 | 0.368 | 0.206 | 0.124 | 0.140 |
| 2.5 | 0.721 | 0.347 | 0.195 | 0.111 | 0.127 |
| 1.25 | 0.434 | 0.237 | 0.158 | 0.109 | 0.130 |
| 0.625 | 0.209 | 0.166 | 0.127 | 0.109 | 0.111 |
| 0.3125 | 0.136 | 0.137 | 0.228 | 0.108 | 0.112 |
| 0.15625 | 0.128 | 0.118 | 0.111 | 0.117 | 0.110 |
| 0.078125 | 0.129 | 0.129 | 0.125 | 0.125 | 0.228 |

Der p1HCE440-recombinant proDer
p1 C4S expressed in E. coli

| | Positive Sera Pool | | | Negative | |
|---|---|---|---|---|---|
| ug/mL | 1:31 | 1:121 | 1:481 | 1:31 | Background |
| 10 | 0.720 | 0.335 | 0.199 | 0.129 | 0.139 |
| 5 | 0.652 | 0.319 | 0.177 | 0.109 | 0.115 |
| 2.5 | 0.734 | 0.338 | 0.175 | 0.108 | 0.126 |
| 1.25 | 0.391 | 0.219 | 0.142 | 0.120 | 0.118 |
| 0.625 | 0.191 | 0.141 | 0.120 | 0.115 | 0.116 |
| 0.3125 | 0.133 | 0.121 | 0.110 | 0.106 | 0.112 |
| 0.15625 | 0.134 | 0.130 | 0.117 | 0.122 | 0.117 |
| 0.078125 | 0.132 | 0.135 | 0.129 | 0.139 | 0.119 |

Der p1HCE448-recombinant proDer
p1C4S C31S C71S expressed in E. coli

| | Positive Sera Pool | | | Negative | |
|---|---|---|---|---|---|
| ug/mL | 1:31 | 1:121 | 1:481 | 1:31 | Background |
| 10 | 0.354 | 0.239 | 0.166 | 0.117 | 0.125 |
| 5 | 0.331 | 0.230 | 0.159 | 0.115 | 0.117 |
| 2.5 | 0.319 | 0.220 | 0.146 | 0.102 | 0.112 |
| 1.25 | 0.251 | 0.180 | 0.127 | 0.102 | 0.108 |
| 0.625 | 0.162 | 0.140 | 0.122 | 0.109 | 0.115 |
| 0.3125 | 0.130 | 0.124 | 0.120 | 0.114 | 0.112 |
| 0.15625 | 0.134 | 0.120 | 0.124 | 0.124 | 0.124 |
| 0.078125 | 0.136 | 0.133 | 0.120 | 0.123 | 0.117 |

Der p1HCE447-recombinant proDer p1C4S C71S expressed in E. coli

| | Positive Sera Pool | | | Negative | |
|---|---|---|---|---|---|
| ug/mL | 1:31 | 1:121 | 1:481 | 1:31 | Background |
| 10 | 0.485 | 0.306 | 0.214 | 0.177 | 0.179 |
| 5 | 0.516 | 0.284 | 0.451 | 0.150 | 0.164 |
| 2.5 | 0.568 | 0.272 | 0.212 | 0.147 | 0.162 |
| 1.25 | 0.376 | 0.236 | 0.190 | 0.146 | 0.152 |
| 0.625 | 0.193 | 0.156 | 0.145 | 0.130 | 0.135 |
| 0.3125 | 0.140 | 0.127 | 0.135 | 0.136 | 0.336 |
| 0.15625 | 0.130 | 0.126 | 0.134 | 0.124 | 0.124 |
| 0.078125 | 0.138 | 0.132 | 0.151 | 0.145 | 0.117 |

The data demonstrate that the Der p 1 containing selected, engineered mutations have a reduced binding affinity for IgE when compared to wt Der p 1.

### Example 6

This example discloses a comparison of the relative IgE binding activity of Der p 1 muteins as measured by competitive ELISA inhibition. The assays measured the ability of fluid phase wt Der p1 or mutated Der p1 to competitively inhibit the binding of IgE to surface bound wt or mutated Der p1.

Each allergen was tested using an individual microtiter plate and each experiment consisted of a series of ELISA's using soluble wt Der p1 as a reference allergen and a series of ELISA's using a soluble mutein. To prepare the plates, all the wells of a microtiter plate were coated as described in section A of Example 5, with the exception that for inhibition assays, the entire plate was coated with a single type of allergen using a coating concentration of 2.5 μg/ml. 50 μl of Tris-HCl Diluent (50 mM Tris-HCl, pH7.5, 2 mM MgCl₂, 145 mM NaCl, 1% BSA, 0.3 mM NaN₃, 0.05% Tween-20) were then added to wells serving as positive controls and 50 μl of negative sera were added to wells assigned to serve as negative controls. The reference allergen (native Der p1) and the test allergen were both diluted to a concentration of 30 μg/ml with Tris-HCl Diluent after which, eight 3-fold serial dilutions were made and 50 μl of each diluted sample added to individual wells of the plate. Lastly, 50 μl of positive sera (diluted 1:40 with Tris-HCl Diluent) were added to the positive control wells and to all wells containing diluted antigen after which the plate was incubated overnight at 4° C. in a humidified chamber. The following day, the wells were washed with Wash Buffer (50 mM Tris, pH 7.5, 145 mM NaCl, 0.3 mM NaN₃, 0.05% Tween-20), the plate blotted dry and 100 μl of biotinylated human Fc receptor (diluted to 10 ng/ml in Tris-HCl Diluent) were added to each well. The plate was incubated at RT for 2 hours after which the wells were washed with Wash Buffer, the plate blotted dry and 100 μl of streptavidin-alkaline phosphatase conjugate (diluted to 125 ng/ml in Tris-HCl Diluent) added to each well. The plate was incubated at RT for 1 hour after which the wells were washed four times with Wash Buffer, the plate blotted dry and 100 μl of p-Nitrophenyl Phosphate Substrate Solution (Moss Inc.) were added to each well. After incubation at RT for 1 hour, the development reaction was stopped by the addition of 50 μl of 20 mM cysteine. The optical density of each well was determined at 405 nM, the results of which are shown below in Table 2. Mutein IgE binding ability is shown as percent inhibition of IgE-allergen binding as compared to a wt Der p1 assay done on the same plate. The Der p1 molecules are numbered in the table as follows:

| | |
|---|---|
| 1620-15 | native Der p1 |
| HCE389 | recombinant wt proDer p1 |
| HCE400 | recombinant Der p1 Δ31-34 |
| HCE413 | recombinant Der p1 Δ31-34 C71S |
| HCE433 | recombinant Der p1 C4S C117S |
| HCE440 | recombinant Der p1 C4S |
| HCE446 | recombinant Der p1 C4S C31S |
| HCE447 | recombinant Der p1 C4S C71S |
| HCE448 | recombinant Der p1 C4S C31S C71S |

TABLE 2
Inhibition of Binding

| Coat | Inhibiting | % Inhibition at Inhibiting Antigen Concentration (uG/mL) | | | | | | | | |
|---|---|---|---|---|---|---|---|---|---|---|
| Antigen | Antigen | 15.0 | 5.0 | 1.667 | 0.556 | 0.185 | 0.062 | 0.021 | 0.007 | 0.002 |
| 1620-15 | 1620-15 | 95 | 94 | 91 | 90 | 82 | 74 | 63 | 50 | 32 |
| 1620-15 | HCE 389 | 74 | 67 | 63 | 51 | 45 | 34 | 26 | 16 | 7 |
| 1620-15 | HCE 446 | 40 | 33 | 30 | 28 | 24 | 18 | 13 | 14 | 11 |
| 1620-15 | HCE 448 | 31 | 24 | 18 | 18 | 14 | 7 | 4 | 6 | 1 |
| 1620-15 | HCE 413 | 61 | 53 | 40 | 38 | 32 | 24 | 14 | 13 | 9 |
| 1620-15 | HCE 440 | 85 | 44 | 26 | 24 | 14 | 9 | 6 | 10 | 6 |
| 1620-15 | HCE 400 | 53 | 42 | 34 | 25 | 14 | 6 | 0 | 2 | 0 |
| 1620-15 | HCE 447 | 84 | 31 | 14 | 12 | 0 | 0 | 0 | 0 | 0 |
| HCE389 | 1620-15 | 88 | 80 | 68 | 66 | 65 | 61 | 54 | 44 | 30 |
| HCE389 | HCE 389 | 89 | 82 | 74 | 69 | 58 | 47 | 31 | 18 | 6 |
| HCE389 | HCE 446 | 41 | 37 | 27 | 24 | 23 | 17 | 8 | 13 | 0 |
| HCE389 | HCE 448 | 32 | 17 | 18 | 10 | 11 | 5 | 0 | 0 | 0 |
| HCE389 | HCE 413 | 42 | 35 | 29 | 28 | 21 | 14 | 8 | 7 | 3 |
| HCE389 | HCE 440 | 38 | 32 | 25 | 24 | 15 | 7 | 1 | 0 | 0 |
| HCE389 | HCE 400 | 44 | 35 | 32 | 31 | 22 | 19 | 15 | 13 | 7 |
| HCE389 | HCE 447 | 38 | 32 | 29 | 25 | 17 | 11 | 1 | 3 | 0 |
| 1620-15 | 1620-15 | 92 | 92 | 90 | 86 | 83 | 75 | 65 | 49 | 34 |
| 1620-15 | HCE 389 | 78 | 71 | 62 | 55 | 46 | 35 | 22 | 22 | 14 |
| 1620-15 | HCE 433 | 39 | 20 | 14 | 18 | 20 | 11 | 3 | 6 | 9 |
| 1620-15 | HCE 446 | 32 | 24 | 15 | 21 | 17 | 5 | 0 | 7 | 5 |
| HCE 389 | 1620-15 | 88 | 81 | 74 | 70 | 69 | 66 | 62 | 50 | 35 |
| HCE 389 | HCE 389 | 92 | 87 | 82 | 74 | 65 | 47 | 36 | 28 | 10 |
| HCE 389 | HCE 433 | 47 | 31 | 27 | 15 | 18 | 6 | 11 | 5 | 2 |
| HCE 389 | HCE 446 | 33 | 30 | 26 | 26 | 14 | 6 | 0 | 7 | 1 |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method of producing a hypoallergenic recombinant mite Group 1 protein, wherein said hypoallergenic recombinant mite Group 1 protein has reduced binding to IgE, but is able to cause proliferation of a T cell that proliferates in response to a native mite Group 1 protein, said method comprising the steps of:
(a) altering a nucleic acid molecule encoding the mite Group 1 protein to produce a variant nucleic acid molecule; and
(b) producing the protein recombinantly from the variant nucleic acid molecule.

2. The method of claim 1, wherein said variant nucleic acid molecule differs from the wild type nucleic acid molecule by alteration of codons for cysteine residues involved in disulfide binding.

3. The method of claim 1, wherein said variant nucleic acid molecule encodes a variant recombinant mite Group 1 protein selected from the group consisting of a pro-form of a variant recombinant mite Group 1 protein and a mature form of a variant recombinant mite Group 1 protein.

4. The method of claim 1, wherein said recombinant mite Group 1 protein is selected from the group consisting of Acarus siro, Aleuroglyphus ovatus, Blomia kulagini, Blomia tropicalis, Chortoglyphus arcuatus, Dennatophagoides farinae, Dermatophagoides microceras, Dermatophagoides pteronyssinus, Euroglyphus maynei, Glycyphagus domesticus, Gohieriafusca, Lepidoglyphus destructor, Psoroptes ovis, Pterolichus obtusus, Sarcoptes scaiei, Tyrophagus longior, and Tyrophagus putrescentiae recombinant mite Group 1 proteins.

5. The method of claim 1, wherein said variant recombinant mite Group 1 protein is a variant of a protein isolated from an organism selected from the group consisting of Dermatophagoides farinae, Dermatophagoides pteronyssinus, and Euroglyphus maynei.

6. The method of claim 1, wherein said variant nucleic acid molecule comprises a nucleic acid sequence encoding an amino acid sequence selected from the group consisting of SEQ ID NO:2, SEQ ID NO:5, SEQ ID NO:8, SEQ ID NO:11, SEQ ID NO: 14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, and SEQ ID NO:44.

7. The method of claim 1, wherein said variant nucleic acid molecule comprises a nucleic acid sequence selected from the group consisting of SEQ ID NO:1, SEQ ID NO:3, SEQ ID NO:4, SEQ ID NO:6, SEQ ID NO:7, SEQ ID NO:9, SEQ ID NO:10, SEQ ID NO:12, SEQ ID NO:13, SEQ ID NO:15, SEQ ID NO:16, SEQ ID NO:18, SEQ ID NO:19, SEQ ID NO:21, SEQ ID NO:22, SEQ ID NO:24, SEQ ID NO:25, SEQ ID NO:27, SEQ ID NO:28, SEQ ID NO:30, SEQ ID NO:31, SEQ ID NO:33, SEQ ID NO:34, SEQ ID NO:36, SEQ ID NO:37, SEQ ID NO:39, SEQ ID NO:40, SEQ ID NO:42, SEQ ID NO:43, and SEQ ID NO:45.

8. A composition comprising a mite Group 1 protein produced in accordance with the method of claim 1 and an excipient.

9. A method to reduce an allergic response to a mite Group 1 protein in a mite-allergic animal, said method comprising administering to said animal a composition of claim 8.

10. A recombinant mite Group 1 protein produced in accordance with claim 1.

11. An isolated nucleic acid molecule comprising a nucleic acid sequence encoding a variant mite group I protein wherein said variant is a hypoallergenic mite group 1 protein wherein said hypoallergenic mite Group 1 protein has reduced binding to IgE, but is able to cause proliferation of a T cell that proliferates in response to a native mite Group 1 protein.

12. The nucleic acid molecule of claim 11, wherein said variant nucleic acid molecule differs from the wild type nucleic acid molecule by alteration of codons for cysteine residues involved in disulfide binding.

13. The nucleic acid molecule of claim 11, wherein said variant nucleic acid molecule encodes a variant recombinant mite Group 1 protein selected from the group consisting of a pro-form of a variant recombinant mite Group 1 protein and a mature form of a variant recombinant mite Group 1 protein.

14. The nucleic acid molecule of claim 11, wherein said variant mite Group 1 protein is selected from the group consisting of Acarus siro, Aleuroglyphus ovatus, Blomia kulagini, Blomia tropicalis, Chortoglyphus arcuatus, Dermatophagoides farinae, Dermatophagoides microceras, Dermatophagoides pteronyssinus, Euroglyphus maynei, Glycyphagus domesticus, Gohieria fusca, Lepidoglyphus destructor, Psoroptes ovis, Pterolichus obtusus, Sarcoptes scaiei, Tyrophagus longior, and Tyrophagus putrescentiae recombinant mite Group 1 proteins.

15. The nucleic acid molecule of claim 11, wherein said variant mite Group 1 protein is a variant of a protein isolated from an organism selected from the group consisting of Dermatophagoides farinae, Dermatophagoides pteronyssinus, and Euroglyphus maynei.

16. An isolated nucleic acid molecule comprising a nucleic acid sequence encoding a protein with an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, and SEQ ID NO:41.

17. An isolated nucleic acid molecule comprising a nucleic acid sequence selected from the group consisting of SEQ ID NO:1, SEQ ID NO:3, SEQ ID NO:4, SEQ ID NO:6, SEQ ID NO:7, SEQ ID NO:9, SEQ ID NO:10, SEQ ID NO:12, SEQ ID NO:13, SEQ ID NO:15, SEQ ID NO:16, SEQ ID NO:18, SEQ ID NO:19, SEQ ID NO:21, SEQ ID NO:22, SEQ ID NO:24, SEQ ID NO:25, SEQ ID NO:27, SEQ ID NO:28, SEQ ID NO:30, SEQ ID NO:31, SEQ ID NO:33, SEQ ID NO:34, SEQ ID NO:36, SEQ ID NO:37, SEQ ID NO:39, SEQ ID NO:40, and SEQ ID NO:42.

18. A recombinant molecule comprising a nucleic acid molecule as set forth in claim 11 operatively linked to a transcription control sequence.

19. A recombinant microorganism comprising a nucleic acid molecule as set forth in claim 11.
